SEQUENCE LISTING (1 sequence)

SEQ ID NO: 1 - DNA, 1050 bp, Escherichia coli
  source 1..1050: mol_type = genomic DNA
atgaagatcaaagctgttggtgcatattccgctaaacaaccacttgaaccgatggatatcacccggcgtgaaccgggaccgaatgatgtcaaaatcgaaatcgcttactgtggcgtttgccattccgatctccaccaggtccgttccgagtgggcggggacggtttacccctgcgtgccgggtcatgaaattgtggggcgtgtggtagccgttggtgatcaggtagaaaaatatgcgccgggcgatctggtcggtgtcggctgcattgtcgacagttgtaaacattgcgaagagtgtgaagacgggttggaaaactactgtgatcacatgaccggcacctataactcgccgacgccggacgaaccgggccatactctgggcggctactcacaacagatcgtcgttcatgagcgatatgttctgcgtattcgtcacccgcaagagcagctggcggcggtggctcctttgttgtgtgcagggatcaccacgtattcgccgctacgtcactggcaggccgggccgggtaaaaaagtgggcgtggtcggcatcggcggtctgggacatatggggattaagctggcccacgcgatgggggcacatgtggtggcatttaccacttctgaggcaaaacgcgaagcggcaaaagccctgggggccgatgaagttgttaactcacgcaatgccgatgagatggcggctcatctgaagagtttcgatttcattttgaatacagtagctgcgccacataatctcgacgattttaccaccttgctgaagcgtgatggcaccatgacgctggttggtgcgcctgcgacaccgcataaatcgccggaagttttcaacctgatcatgaaacgccgtgcgatagccggttctatgattggcggcattccagaaactcaggagatgctcgatttttgcgccgaacatggcatcgtggctgatatagagatgattcgggccgatcaaattaatgaagcctatgagcgaatgctgcgcggtgatgtgaaatatcgttttgttatcgataatcgcacactaacagactga

(19) United States
(12) Patent Application Publication
(10) Pub. No.: US 2024/0158818 A1
(43) Pub. Date: May 16, 2024

(54) Recombinant Escherichia coli producing xylitol from xylose, method for preparing the same, and uses thereof

(71) Applicant: MYONGJI UNIVERSITY INDUSTRY AND ACADEMIA COOPERATION FOUNDATION, Yongin-si (KR)

(72) Inventors: Wook Jin Chung, Seoul (KR); Angelo Banares, Yongin-si (KR); Won Keun Lee, Yongin-si (KR)

(21) Appl. No.: 18/450,612

(22) Filed: Aug. 16, 2023

(30) Foreign Application Priority Data
Aug. 16, 2022 (KR) 10-2022-0102333

(51) Int. Cl. C12P 7/18; C12N 1/20; C12N 9/04; C12N 15/70

(52) U.S. Cl. CPC C12P 7/18; C12N 1/205; C12N 9/0006; C12N 15/70; C12N 2500/34; C12N 2500/35; C12Y 101/01002

(57) ABSTRACT

The present disclosure relates to a recombinant E. coli strain that produces xylitol from xylose, a method for preparing the same, and a use thereof. Specifically, according to an embodiment of the present disclosure, there is provided a method for producing xylitol, wherein the method includes: culturing the recombinant E. coli strain transformed with an expression vector including a gene encoding a YahK enzyme and the recombinant E. coli strain on a substrate containing xylose (stage 1); and obtaining the xylitol from the culture cultured in stage 1 (stage 2).

### CROSS-REFERENCE TO RELATED APPLICATION

This application claims priority to and the benefit of Korean Patent Application No. 10-2022-0102333 filed in the Korean Intellectual Property Office on Aug. 16, 2022, the entire content of which is incorporated herein by reference.

### INCORPORATION OF SEQUENCE LISTING

The sequence listing that is contained in the file named “Sequence_Listing_M347.0002US1.xml” which is 3 kilobytes as measured in Microsoft Windows operating system and was created on Jan. 10, 2024, and is filed electronically herewith and incorporated by reference.

### TECHNICAL FIELD

The present disclosure relates to a recombinant E. coli strain that produces xylitol from xylose, a method for preparing the same, and a use thereof.

### BACKGROUND

Xylitol is in high demand worldwide, primarily because of its widespread use in the food and pharmaceutical industries. In addition, xylitol may be utilized as a building block molecule for the production of high value-added chemicals.

Currently, the industrial production of such xylitol is made through chemical synthesis involving the catalytic hydrogenation of xylose. However, when xylitol is produced by chemical reaction, there are issues such as yield reduction due to side reaction products, high energy consumption, and high temperature and high pressure conditions. Accordingly, the production of biotechnological xylitol has emerged as a promising low-cost, sustainable alternative to chemical synthesis.

Xylitol may be created naturally by microorganisms (for example, molds, yeasts and bacteria). Natural xylitol producers mainly include yeast species such as Pichia stipitis, Hansenula polymorpha TCC 34438, Kluyveromyces marxianus IMB5, Meyerozyma guilliermondii, Debaryomyces hansenii, Candida guilliermondii, and Kluyveromyces marxianus (CCA 510). In yeast and other xylitol-producing microorganisms, xylose is catalyzed to xylitol via NADH or NADPH-dependent xylose reductase (XR). Xylitol is an intermediate extruded out of cells during xylose metabolism through the oxidoreductase pathway. Extrusion of xylitol is due to a cofactor imbalance between NADPH-dependent XR and NAD+-dependent xylitol dehydrogenase (XLH). The latter catalyzes the reaction of xylitol to xylulose, which is phosphorylated to xylulose-5-phosphate and eventually assimilated into central carbon metabolism (CCM).

Consumption of xylitol by natural producers makes it difficult to produce xylitol in high yield. Accordingly, further engineering of natural producers is necessary to improve xylitol accumulation and avoid xylitol assimilation into CCM. However, some yeast species, such as P. stipites and M. guilliermondii, are considered unsafe, limiting their use in the food industry. These natural producers are difficult to genetically engineer due to limited genetic information and customized genetic engineering tools.

The use of E. coli as a host for xylitol production has the advantages of simple culture conditions and rapid recombinant protein expression. However, xylitol production in E. coli requires insertion of heterologous XR, and not all heterologous XRs, especially yeast-derived XRs, show optimal expression in bacterial strains. Compatible and having optimal expression are native or endogenous genes often found in host strains. However, no endogenous enzyme catalyzed by xylitol has been known to date.

### SUMMARY

An aspect of the present disclosure is directed to providing a recombinant E. coli strain (transformed E. coli strain) capable of producing xylitol from xylose by expression of an endogenous enzyme.

Another aspect of the present disclosure is directed to providing a method for producing the recombinant E. coli strain.

Another aspect of the present disclosure is directed to providing a method for producing xylitol from xylose using the recombinant E. coli strain.

As a result of intensive research efforts to overcome the issues of the related art, the present inventors have found that plasmid-based expression of the endogenous YahK gene encoding NADPH-dependent aldehyde reductase (ALR) in E. coli enables xylitol production. The present disclosure is the first to identify an endogenous enzyme called YahK as a catalyst for xylitol production. In addition, in contrast to the existing knowledge that the xylose isomerase pathway (XIP) is the only xylose metabolic pathway in wild-type E. coli, the present disclosure further increased productivity of xylitol by using a strain blocking the pathway.

According to one aspect of the present disclosure, there is provided an expression vector including a gene encoding a YahK enzyme.

As used herein, the term “expression vector” refers to a genetic construct including essential regulatory elements such as a promoter so that a target gene may be expressed in a suitable host cell. According to an embodiment of the present disclosure, the expression vector may be a plasmid.

According to an embodiment of the present disclosure, the gene may have a nucleotide sequence of SEQ ID NO: 1, but considering the degeneracy of the genetic code, the gene may be a gene having 80% of homology, preferably 85% of homology, more preferably 90% of homology, and most preferably 95% of homology with the nucleotide sequence represented by SEQ ID NO: 1.

According to an aspect of the present disclosure, there is provided a transformant transformed with an expression vector including a gene encoding the YahK enzyme.

According to an embodiment of the present disclosure, the transformant may have an ability to produce xylitol from xylose.

According to an embodiment of the present disclosure, the transformant may have a blocked xylose isomerase pathway, and xylitol productivity from xylose may be further increased by using a transformant in which the xylose isomerase pathway is blocked as described above.

According to an embodiment of the present disclosure, the transformant may be Escherichia coli, for example, E. coli W3310. In addition, the transformant may be a recombinant E. coli strain in which the YahK enzyme is overexpressed and the xylose isomerase pathway is blocked. According to an embodiment of the present disclosure, the transformant may be E. coli W3310 ΔxylAB pTRCHIS2A-yahK of strain accession number KCTC15024BP.

According to another aspect of the present disclosure, there is provided a method for producing xylitol, wherein the method includes: culturing a transformant transformed with the expression vector including the gene encoding the YahK enzyme in a substrate containing xylose (stage 1); and obtaining the xylitol from the culture cultured in stage 1 (stage 2).

The transformant may refer to what has been described in relation to the transformant above, and may preferably be Escherichia coli, for example, E. coli W3310. Preferably, the transformant may be a transformant overexpressing the YahK enzyme and blocking the xylose isomerase pathway, more preferably E. coli W3310 ΔxylAB pTRCHIS2A-yahK of strain accession number KCTC15024BP.

As a method for culturing the transformant of an embodiment of the present disclosure, a conventional method used for culturing a host may be used. As the culture method, any method used for culturing ordinary microorganisms, such as batch, fluidized batch, continuous culture, reactor, etc., may be used. As a medium for culturing a transformant obtained by using a bacterium such as E. coli as a host, a complete medium or a synthetic medium such as LB medium and NB medium may be exemplified.

Carbon sources are necessary for the growth of microorganisms, and include, for example, saccharides such as glucose, fructose, sucrose, maltose, galactose, and starch; lower alcohols such as ethanol, propanol, and butanol; polyhydric alcohols such as glycerol; organic acids such as acetic acid, citric acid, succinic acid, tartaric acid, lactic acid, and gluconic acid; and fatty acids such as propionic acid, butanoic acid, pentanoic acid, hexanoic acid, heptanoic acid, octanoic acid, nonanoic acid, decanoic acid, undecanoic acid, and dodecanoic acid. According to an embodiment of the present disclosure, the substrate in stage 1 may further contain glycerol. According to an embodiment of the present disclosure, the glycerol may be contained in an amount of 0.2 to 0.8% (v/v).

According to an aspect of the present disclosure, there is provided a composition for producing xylitol including the aforementioned transformant. The composition of an embodiment of the present disclosure may include, for example, a polypeptide, broth, and cell lysate suitable for culturing the transformant to produce xylitol.

According to an embodiment of the present disclosure, a recombinant E. coli strain (E. coli W3110ΔxylAB pTRCHIS2A-yahK) overexpressing the YahK enzyme and blocking the xylose isomerase mechanism was developed and succeeded in biosynthesizing xylose into xylitol. According to an embodiment of the present disclosure, xylitol may be produced from xylose in a one-stage biosynthetic reaction. In addition, since there is no production of by-products and the reaction occurs at room temperature, issues of the related art due to chemical reactions, such as yield reduction due to side reaction products and high temperature conditions, may be solved.

### BRIEF DESCRIPTION OF THE DRAWINGS

FIG. 1 is a plasmid map of PTrcHis2A-yahK.

FIG. 2 is a metabolic pathway in which xylitol and ethylene glycol (EG) are produced from D-xylose in E. coli W3110. The genes typed in red are overexpressed, and metabolites in green and blue are a portion of the xylose isomerase pathway and the Dahms pathway, respectively.

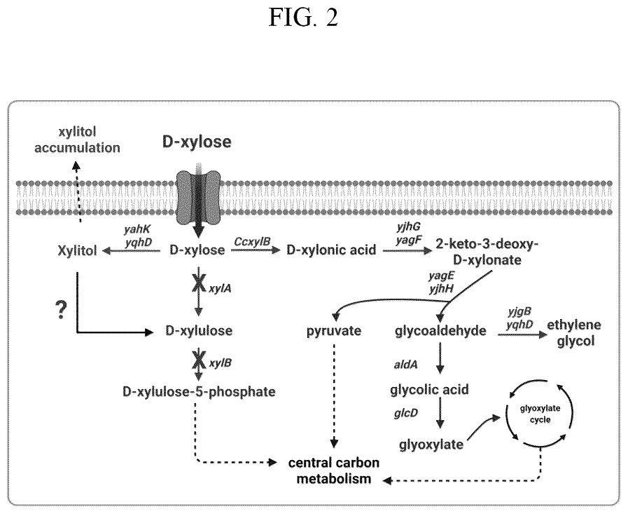

FIG. 3 shows the results of screening candidate ALR genes (yahK, yjgB, and yqhD). In E. coli strains overexpressing yahK and yqhD, after 72 hours of fermentation, each of 1.89 g/L and 0.84 g/L of xylitol was detected from 4 g/L D-xylose, while only minimal amounts of EG were produced.

FIG. 4 is a GC-MSD and HPLC spectrum for identifying whether xylitol is present in a specimen. (a) is the fragmentation pattern of commercially available xylitol, (b) is the fragmentation pattern of xylitol from the database, (c) is the fragmentation pattern of the specimen, and (d) is the HPLC retention time of the specimen.

FIG. 5 shows the results of enzyme characteristic analysis for YahK. a is a graph showing the relative activity of YahK in different sugars, b shows the relative activity of YahK with temperature change, and c shows the relative activity of YahK with pH change.

FIG. 6 shows the effect of xylose metabolic pathways on xylitol production. Xylose reductase (Yahk) was abbreviated as XR, and the Dahms pathway and the xylose isomerase pathway were abbreviated as DP and XIP, respectively. (a) XOL1 strain with XR and DP, (b) XOL2 strain with XR+XIP

FIG. 7 is a graph showing the results of producing xylitol from D-xylose using different cosubstrates. a is the metabolic profile of an XOL3 strain in the presence of 2.5 g/L glucose, b is the metabolic profile of the XOL3 strain in the presence of 0.4% glycerol (v/v), and c is a graph showing the results of producing xylitol in the XOL3 strain in the presence of various concentrations of glycerol.

FIG. 8 is a graph showing changes in concentrations of biomass, D-xylose, and xylitol during culturing of E. coli strains in a 5 L fermentor. a is the result of using the XOL3 strain with both XR and XIP, and b is the result of using the XOL2 strain with only the XR. Herein, XIP is an abbreviation for xylose isomerase pathway and XR is an abbreviation for xylose reductase (Yahk). The green vertical line indicates the addition of glycerol.

FIG. 9 is a graph showing changes in concentrations of biomass, D-xylose, glycerol, and xylitol over time during culturing of XOL2 strain in a 500 mL baffle flask induced by varying the concentration of IPTG.

FIG. 10 shows experimental procedures and results of producing xylitol from D-xylose using a whole-cell biocatalyst (WBC). a shows the process of producing xylitol using WBCs, b is a graph showing the results of using 7.5 g/L glucose as a cosubstrate with varying WBC cell densities, and c is a graph showing the results using 5% glycerol (v/v) with varying WBC cell densities.

FIG. 11 is a graph showing the amount of xylitol produced after fermentation for 24 hours using a whole cell biocatalyst having an OD of 10 at various IPTG concentrations, where 30 g/L xylose was used.

### DETAILED DESCRIPTION

Hereinafter, the present disclosure will be described in more detail through examples. Objects, features and advantages of the present disclosure will be easily understood through the following examples. The present disclosure is not limited to the embodiments described herein and may be embodied in other forms. The embodiments introduced herein are provided to sufficiently convey the spirit of the present disclosure to those skilled in the art to which the present disclosure pertains. Accordingly, the present disclosure should not be limited by the following examples.

[Construction of Strains and Plasmids]

All strains and recombinant plasmids used in this experiment are listed in Table 1 below. Previously constructed strains were reused and recombinant plasmids were transformed using a one-step TSS (transformation and storage solution) protocol [Chung C T, Niemela S L, Miller R H. One-step preparation of competent Escherichia coli: transformation and storage of bacterial cells in the same solution. Proc Natl Acad Sci USA. 1989 April; 86(7):2172-5. doi: 10.1073/pnas.86.7.2172.].

TABLE 1
Strains and plasmids used in this experiment

| Strains/Plasmids | Features/Genotype | Reference |
|---|---|---|
| Plasmids | | |
| pTrcHis2A | pBR322 derivative; Trc promoter; rrnB anti- | Addgene |
| | terminator; Ap^{r} | |
| pKMX | pBBR1-MCS2 derivative; tac promoter, R. | Valdehuesa et al., 2014 |
| | eutropha PHA biosynthesis genes | |
| | transcription terminator; Km^{r} | |
| pTrcHis2A_yahK | pTrcHis2A derivative; P_{trc}-yahK | Cabulong et al., 2017 |
| pTrcHis2A_yjgB | pTrcHis2A derivative; P_{trc}-yjgB | Cabulong et al., 2017 |
| pTrcHis2A_yqhD | pTrcHis2A derivative; P_{trc}-yqhD | Cabulong et al., 2017 |
| Strains | | |
| EG3 | Ecl; pKMX and pTrcHis2A_yjgB | Cabulong et al., 2017 |
| EG4 | Ecl; pKMX and pTrcHis2A_yqhD | Cabulong et al., 2017 |
| XOL0 | Ecl; pKMX and pTrcHis2A | Cabulong et al., 2017 |
| XOL1 | Ecl; pKMX and pTrcHis2A_yahK | Cabulong et al., 2017 |
| XOL2 | Wild type E. coli W3110 with | Present disclosure |
| | pTrcHis2A_yahK | |
| XOL3 | E. coli W3110 ΔxylAB; pTrcHis2A_yahK | Present disclosure |

* Valdehuesa et al., 2014 = Valdehuesa K N G, Liu H, Ramos K R M, Park S-J, Nisola G M, Lee W-K, Chung W J. Direct bioconversion of D-xylose to 1,2,4-butanetriol in an engineered Escherichia coli. Process Biochem. 2014 January; 49(1): 25-32. doi: 10.1016/j.procbio.2013.10.002
* Cabulong et al., 2017 = [Cabulong R B, Valdehuesa K N, Ramos K R, Nisola G M, Lee W K, Lee C R, Chung W J. Enhanced yield of ethylene glycol production from d-xylose by pathway optimization in Escherichia coli. Enzyme Microb Technol. 2017 February; 97: 11-20. doi: 10.1016/j.enzmictec.2016.10.020.]

### General Experimental Method

Purification of YahK

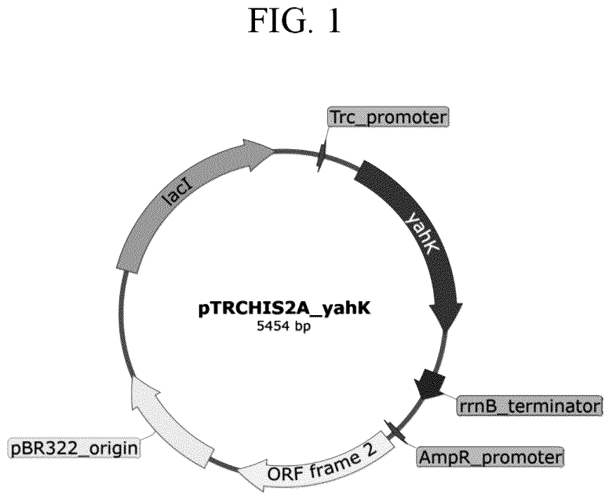

pTrcHis2A having a yahK gene including a 6×-histidine tag at the C-terminus was transformed into E. coli W3110 (DE3) (see FIG. 1). The produced strain was grown in 100 mL LB medium for up to 8 hours. Cells were then collected by centrifugation at 4000 rpm for 15 minutes at 4° C. After centrifugation, His-tag YahK was extracted and purified according to the protocol of a Protino Ni-TED 2000 packed column (Macherey-Nagel, Germany). The purified YahK was visualized in a polyacrylamide gel and concentration was measured using Bradford assay (Bradford 1976). Then, the purified Yahk was dialyzed using 50 mM TRIS-HCl.

Shake-Flask Fermentation

The modified M9 (MM9) medium was used, including 1×M9 medium with 1 g/L peptone, 0.5 g/L yeast extract and 1.0 mM MgSO4. For shake flask fermentation, a 300 mL Erlenmeyer flask containing 100 mL of MM9 with appropriate antibiotics and specific sugar substrate(s) was used. Each flask was inoculated with 1 mL of the overnight culture and incubated for 72-96 hours at 37° C. with agitation at 180 rpm. On the other hand, a similar method was applied to scale-up shake flask fermentation except that a 500 mL baffled Erlenmeyer flask containing 200 mL of MM9 with appropriate antibiotics and sugar substrate inoculated with 2 mL of the overnight culture was used. 1 mL samples were collected at designated time intervals to monitor growth and metabolite concentrations as described in previous studies (Cabulong et al., 2017, 2018).

Checking of Xylitol

Xylitol accumulation was identified using GC-MSD and HPLC analysis. GC-MSD analysis was performed using the method described in Valdehuesa et al. (2014) with minor modifications. Briefly, 500 μL of clarified fermentation broth was acidified by adding 80% (v/v) of sulfuric acid. The acidified solution was lyophilized and dissolved in 250 μL of pyridine. The pyridine mixture was then derivatized by adding 250 μL of N,O-bis(trimethylsilyl)trifluoroacetamide (BSTFA) and incubated at 70° C. for 30 minutes. The prepared samples were then analyzed using an HP 6890 GC/HP5973 Mass Selective Detector GC-MSD. A 1.0 μL sample was injected at a 50:1 split ratio with a helium carrier gas at a flow rate of 2.0 mL/min. Then, the GC-MS chromatograms of the fermentation samples were compared with an existing in-house standard database and a database of commercially available xylitol. Meanwhile, HPLC analysis of the fermentation broth was compared with standard xylitol using the method described above.

Bioreactor Experiment

Bioreactor studies were performed in a 5 L bioreactor with 2 L of MM9 supplemented with specific antibiotic(s) and sugar substrate(s). 2 mL of starter cultures collected from colonies on LB-Agar medium and cultured in 5 mL LB medium were transferred to flasks with 100 mL LB medium. The flask was incubated overnight at 37° C. with stirring at 180 rpm. Cells were harvested by centrifugation and resuspended in 100 mL of MM9 prior to inoculation into the bioreactor. Fermentation parameters were controlled at 37° C., pH 7, 250 rpm agitation and airflow of 0.5 vvm. The pH was adjusted using 1N NaOH and 4N H2SO4 solution.

Whole-Cell Biocatalyst Experiment

Cells were reanimated in an LB-Agar medium and incubated for 12 hours at 37° C. Colonies from LB-agar were inoculated into 15 ml of conical tubes containing 5 ml of an LB medium and cultured for 12 hours at 37° C. with stirring at 180 rpm. 2 mL of the starter culture was then transferred to a 500 mL baffled Erlenmeyer flask containing 200 mL of an LB medium and appropriate antibiotics. Cultures were incubated according to the conditions mentioned above. IPTG was added when the optical density of the culture reached 0.3-0.4 at 600 nm for induction. After 12 hours, cells were harvested by centrifugation at 4° C. and washed twice with 50 mM of Tris-HCl buffer (pH 7.0). Cells were then resuspended in an MM9 medium and specific amounts of cells were inoculated into 500 mL of baffled Erlenmeyer flasks with 200 mL of an MM9 medium containing specific sugar substrate(s) and antibiotics to adjust the OD at 600 nm. After 24 hours of incubation, a 1 ml sample was collected for analysis.

### [Experimental Example 1] Identification of Endogenous YahK Enzyme for Xylitol Production

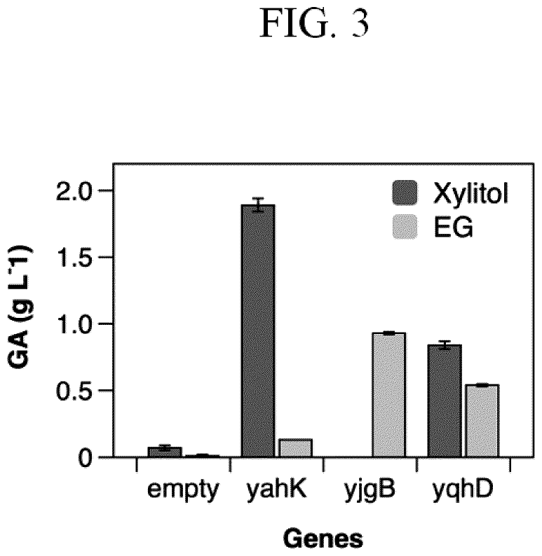

Optimization of ethylene glycol (EG) production in E. coli strain W3110 via the Dahms pathway requires overexpression of an efficient aldehyde reductase (ALR) gene that catalyzes the conversion of glycoaldehyde to ethylene glycol. Accordingly, candidate ALR genes yahK, yjgB and yqhD were screened to optimize EG production in E. coli. Each gene amplified from the genome of E. coli strain W3110 was cloned into the pTRCHIS2A vector downstream of the trc promoter. Unexpectedly, 1.89 g/L and 0.84 g/L of xylitol were detected from 4 g/L D-xylose in E. coli strains overexpressing yahK and yqhD, respectively, after 72 hours of fermentation, but only minimal amounts of EG were produced (FIG. 3). EG3 and control strains were unable to produce xylitol, suggesting that low or non-existent genomic expression of YahK may render xylose insufficient for conversion to xylitol.

### [Experimental Example 2] Checking of Xylitol

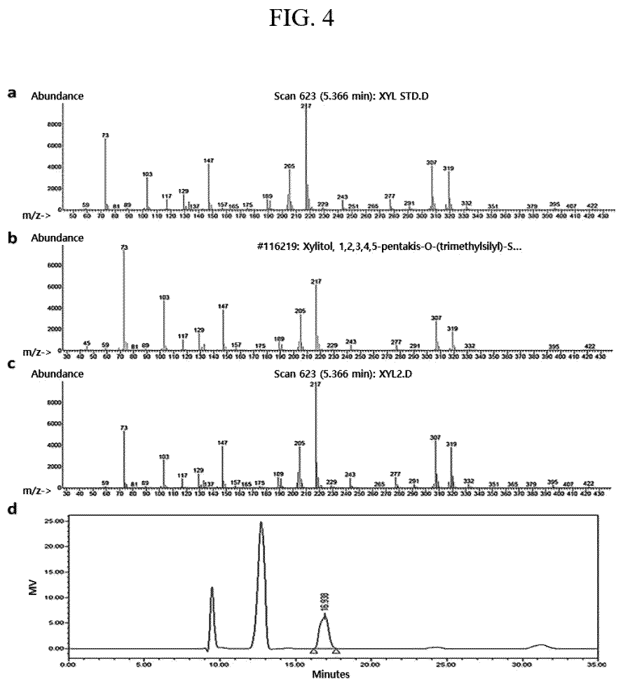

Subsequently, xylitol produced in XOL1 and EG3 was further checked through GC-MSD and HPLC analysis. Briefly, the peak at 16.938 minutes detected through HPLC analysis of the fermentation specimen and the fragmentation pattern observed through GC-MSD analysis were the same peaks as commercially available xylitol and the fragmentation pattern of xylitol, identifying from the database that the product was xylitol (see FIG. 4A-C). These results suggest that overexpression of yahK and yqhD may induce xylitol production in E. coli and that yahK is the most suitable gene for xylitol production (FIG. 1).

### [Experimental Example 3] Enzyme Characteristic Analysis Experiment of YahK

The xylose reductase activity of YahK was measured by monitoring the oxidation of NADPH at 340 nM with minor modifications to the procedure of Sche and Horecker (1996). A total volume of 1.0 mL was prepared containing 100 mM of D-xylose, 1.15 mM of NADPH, 50 mM of Tris-HCl (pH 7.5), and enough enzyme to create an absorbance change between 0.02 and 0.1 ΔE per minute at 37° C. Specific activity is expressed in units per milligram of protein (U/mg protein), where unit (U) is defined as micromol NAD(P)H reduced or oxidized per minute. The optimal pH for YahK was determined at various pH conditions using the following buffers. 50 mM of citrate (pH 3.0-6.0), 50 mM of Tris-Cl (pH 7.0-8.0), and 50 mM of glycine-NaOH (pH 9.0-10.0) at 37° C. The influence of temperature was measured in 50 mM of Tris-Cl (pH 7.5) at various temperatures (20, 30, 40, 50 and 60° C.) using the same mixture. In addition, substrate specificity of YahK for other sugars was performed using the conditions mentioned. Kinetic parameters of xylose reductase relative to D-xylose were determined using 1-100 mM of xylose under the conditions described above.

Substrate Specificity of YahK

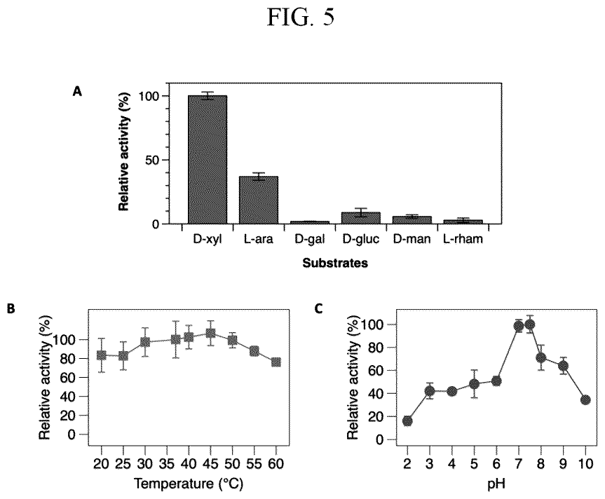

The activity of YahK was tested in the presence of several different sugars. The results are graphically shown in FIG. 5A. According to FIG. 5A, YahK exhibited high activity against D-xylose and activity against L-arabinose, but did not show activity against C6 sugar. As in other xylose reductases, it may be understood that YahK may catalyze the conversion of L-arabinose to L-arabinitol. The presence of this L-arabinitol may lead to purification issues, especially in fermentation media where mixtures of sugars derived from lignocellulose are present for the production of xylitol.

Activity Depending on Temperature and pH

YahK activity was tested according to temperature or pH change, and the results are shown in B and C of FIG. 5, respectively. It may be understood from B and C of FIG. 5 that in terms of temperature and pH, YahK exhibits optimal activity at 45° C. and a pH of 7-8, respectively.

### [Experimental Example 4] Synergistic Effect by Xylose Metabolic Pathway

Removal of the natural xylose pathway in E. coli to alter the xylose metabolic pathway destabilizes access to the pentose phosphate pathway, thus impeding carbon flow towards the formation of precursors important for cofactors, cell metabolism, cell maintenance, redox homeostasis and regulation of oxidative stress, resulting in lower yields of target products. YahK of D-xylose enhanced xylitol production through a synergistic action with the xylose isomerase pathway and the Dahms pathway.

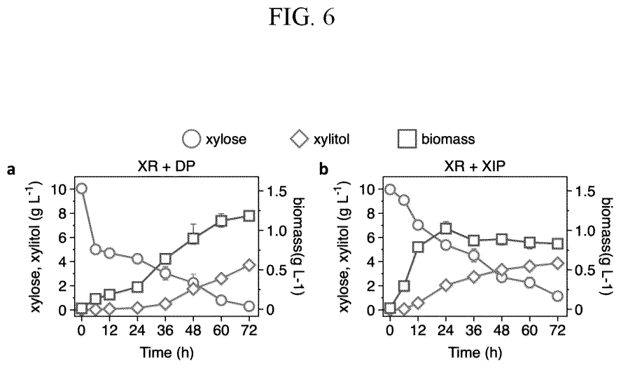

The XOL1 strain showed rapid consumption of xylose within 6 hours, then slowed down, while xylitol production started after 24 hours (FIG. 6A). Rapid xylose consumption may be due to overexpression of the xdh gene, which assimilates xylose into the Dahms pathway, and slow xylose consumption exceeding 6 hours may be due to metabolic load. Nonetheless, xylose was produced with 3.6 g/L of xylitol completely consumed after 72 hours of fermentation, which means that it was utilized for both growth and xylitol production.

The XOL2 strain in which XIP was intact and XR was overexpressed showed an increase in xylitol production and a slow and constant decrease in xylose over time (FIG. 6B).

The XOL2 strain produced 4 g/L of xylitol, showing higher productivity than the XOL1 strain, and as expected, rapid biomass accumulation peaking at 24 hours was observed (FIG. 6A-B). On the other hand, the XOL1 strain showed a long lag phase and gradual biomass increase until the end of fermentation, and the XOL1 strain produced higher biomass, albeit at a lower rate. XOL1 slow biomass formation may be due to low ATP consumption.

Overall, the low xylitol titer in the engineered strains are due to the presence of a xylose pathway that competes with xylose as the sole carbon source. It is also noteworthy that even when additional carbon sources are available, it is still impossible to obtain high concentrations of xylitol due to the presence of other xylose pathways.

### [Experimental Example 5] Addition of Cosubstrate

To increase xylitol titers from xylose, the natural xylose isomerase pathway was blocked to allow xylose to be metabolized only for xylitol production. The strain was constructed by transforming pTRCHIS2A-yahK into an ECI strain to create the XOL3 strain. Since xylose is only used for xylitol production, additional substrates are required to support the production of cofactors or precursors required for growth and cell maintenance of the engineered strain. The supply of NADPH, an important cofactor for YahK to catalyze the conversion of D-xylose to D-xylitol, also comes from cosubstrates. Glycerol and glucose were compared as potential cosubstrates for xylitol production.

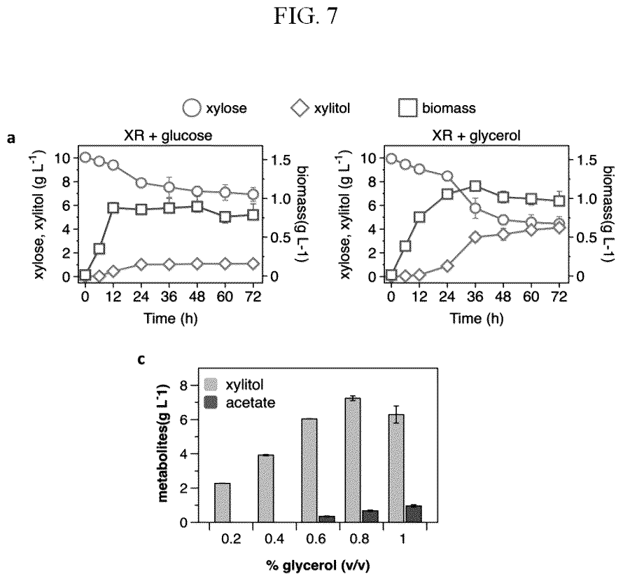

Briefly, 0.4% v/v of glycerol or 2.5 g/L of glucose was added to an MM9 medium containing 10 g/L of D-xylose. As shown in FIG. 6A-B, the biomass formation of engineered strains increased rapidly in the presence of glycerol or glucose. Xylose consumption and conversion to xylitol was very slow in the presence of glucose compared to glycerol, which may be attributed to carbon catabolite repression (CCR). A significant decrease in xylose concentration and an increase in xylitol production were observed upon depletion of glucose and glycerol at 24 and 36 hours, respectively (FIG. 7A-B). The addition of glycerol in the tested mixture produced 4.11 g/L of xylitol after 72 hours of fermentation, which is higher than 1.0 g/L of xylitol produced in the presence of glucose (FIG. 7A). This means that glycerol is a more suitable cosubstrate for xylitol production. As a result, increasing glycerol concentrations achieved high xylitol production reaching up to 7.34 g/L after 60 hours of fermentation in the presence of 0.8% of glycerol (v/v) (FIG. 7C). On the other hand, as the glycerol concentration increases, the production of xylitol also increases, but it is saturated when the glycerol concentration exceeds 0.8% (v/v), and acetate is formed when the glycerol concentration is equal to or greater than 0.6% (v/v) (FIG. 7C). Accordingly, glycerol concentrations of 0.5-0.6% (v/v) were used for subsequent fermentations to avoid acetate formation. Glycerol is cheaper than glucose and has shown promising results in terms of biomass yield and product titer in E. coli cultivation.

### [Experimental Example 6] Scale-Up and Fed-Batch Fermentation

The XOL2 and XOL3 strains underwent scale-up and fed-batch fermentation in 5-L fermentors, respectively. In this experiment, IPTG was not added to rule out whether the periodic addition of glycerol and the presence of the xylose isomerase pathway in E. coli strains might increase xylitol production.

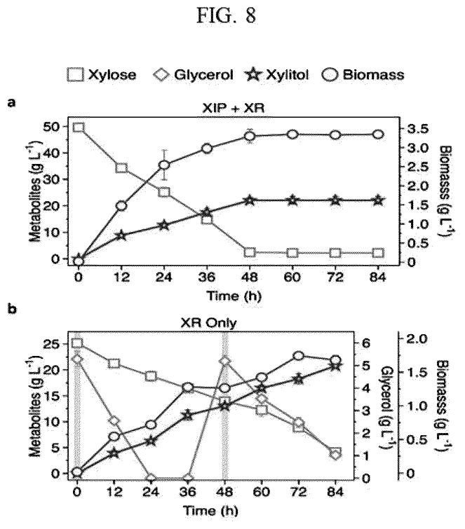

In the case of the XOL2 strain (in other words, a strain having a xylose isomerase pathway, E. coli W3310 pTRCHIS2A-yahK), it was produced from 22.04 g/L of xylitol within 48 hours from 50 g/L of D-xylose, reaching 44% of the theoretical yield with a productivity of 0.61 g/L/h (FIG. 8A).

The periodic supply of 0.5% (v/v) of glycerol every 48 hours also tested in the XOL3 strain (in other words, a strain deficient in the xylose isomerase pathway, E. coli W3310 ΔxylAB pTRCHIS2A-yahK). After 84 hours, 20.77 g/L of xylitol was produced from 25 g/L of xylose, leaving a residual 4 g/L of xylose (FIG. 8B). This reached a theoretical yield of 83.08% with a productivity of 0.47 g/L/h. However, the timing of glycerol addition was inappropriate, as shown by the glycerol consumed after 24 hours (FIG. 7B).

The very high xylitol titers and yields created after fed-batch fermentation may be attributed to the deletion of xylB in the XOL2 strain, which prevents accumulation of the toxic intermediate xylitol-5-phosphate. XylB of E. coli is known to phosphorylate xylitol to xylitol phosphate, inhibiting the growth of E. coli on D-xylose and reducing xylitol production (Akinterinwa and Cirino, 2009).

### [Experimental Example 7] Xylitol Production in E. coli W3310 Ä xylAB pTRCHIS2A-yahK by Varying Concentration of IPTG

As may be understood in the production of xylitol from D-xylose, yahK expression is possible even in the absence of an inducer. However, since yahK expression is located downstream of the IPTG-inducible trc promoter, the addition of IPTG may substantially increase xylitol production in the XOL3 strain. However, too much protein may cause toxicity and metabolic constraints on the host, so it is necessary to determine the appropriate amount of IPTG to increase the YahK concentration at an appropriate level.

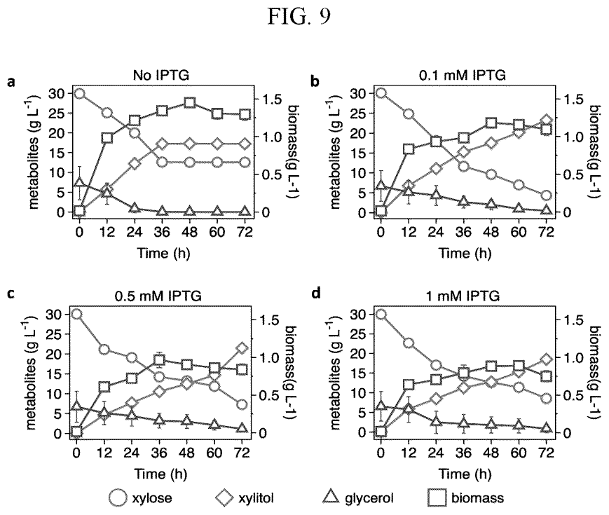

E. coli W3310 ΔxylAB pTRCHIS2A-yahK was cultured in M9 minimal medium with 30 g/L of xylose and 0.6% v/v of glycerol. Among the tested IPTG concentrations, those without IPTG (FIG. 9A) showed relatively high biomass compared to those induced with various concentrations of IPTG. The reduction in biomass may be due to the cytotoxic effect of IPTG addition. In addition, specimens induced with 0.1 mM of IPTG produced the highest amount of 23.27 g/L f xylitol, when compared to 0.0 mM of IPTG, 0.5 mM of IPTG and 1 mM of IPTG produced 17.24 g/L, 21.19 g/L, and 18.05 g/L of xylitol, respectively (FIGS. 9A-D). High xylitol production induced with minimal IPTG reduces production costs, making the strain economically viable.

### [Experimental Example 8] Xylitol Production Through Whole Cell Biocatalyst

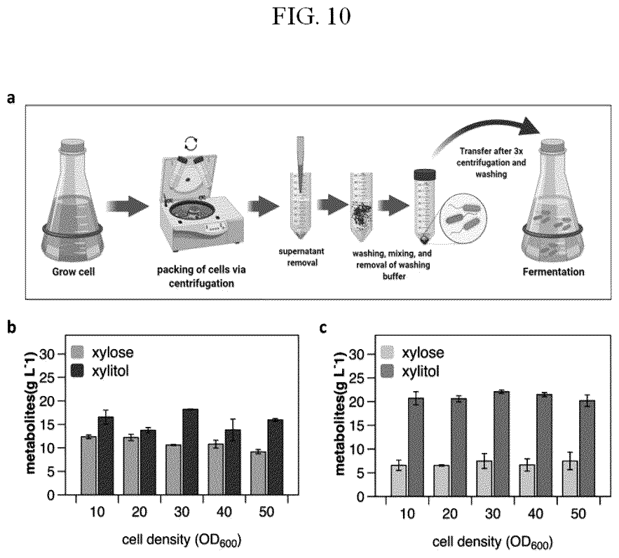

Xylitol production through whole cell biocatalyst (WBC) has many advantages over cell-free biosynthesis and general fermentation processes. Unlike cell-free biosynthesis, enzyme purification is not required and the addition of exogenous cofactors is not required. This is because cells have unique machinery for cofactor production and recycling, making the process economically feasible (FIG. 10A). On the other hand, since the general fermentation process has lower productivity and yield than the WBC method, xylitol production through the WBC was applied.

Both glucose and glycerol were tested as cosubstrates for xylitol production (FIG. 10). The addition of cosubstrates is required to provide reducing equivalents and energy for the WBC. Different concentrations of xylitol were produced using 7.5 g/L of glucose or 5% of glycerol (v/v) at different cell densities (absorbances of 10, 20, 30, 40, and 50 at OD600). For glucose and glycerol, high xylitol titers of 22.08 g/L and 18.20 g/L were produced after 24 hours of culturing when OD600 became 30, suggesting that glycerol is a more promising cosubstrate than glucose for WBCs (FIG. 10B-C). Accordingly, in subsequent experiments, glycerol was used as a cosubstrate.

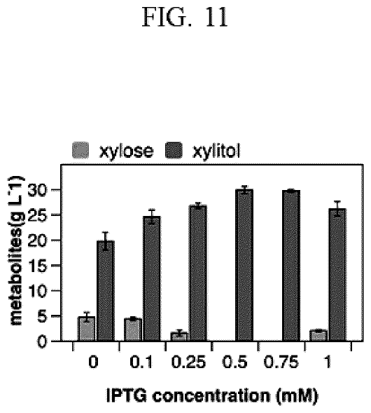

In the next experiment, it has been tested whether reducing the cosubstrate at various IPTG concentrations using 1% of glycerol (v/v) would give the same or better results. To this end, the IPTG concentration was varied between 0 mM and 1 mM (FIG. 11). Of the IPTG concentrations, 0.5 mM and 0.75 mM showed the highest productivity at 24 hours with 30 g/L xylitol (theoretical yield 100%).

Although the above has been described with reference to preferred embodiments of the present disclosure, those skilled in the art will understand that various modifications and changes may be made without departing from the spirit and scope of the present disclosure as described in the claims below.

Depository Institution: Korean Collection for Type Cultures (KCTC) of Korea Research Institute of Biotechnology and Bioscience

Accession No.: KCTC15024BP

Deposit Date: 20220701

1. An expression vector comprising a gene encoding a YahK enzyme.

2. The expression vector of claim 1, wherein the gene has a nucleotide sequence of SEQ ID NO: 1.

3. The expression vector of claim 1, wherein the expression vector is a plasmid.

4. A transformant transformed with the expression vector of claim 1.

5. The transformant of claim 4, wherein the transformant has a blocked xylose isomerase pathway.

6. The transformant of claim 4, wherein the transformant is Escherichia coli.

7. The transformant of claim 4, wherein the transformant is E. coli W3310 ΔxylAB pTRCHIS2A-yahK of strain accession number KCTC15024BP.

8. The transformant of claim 4, wherein the transformant has an ability to produce xylitol from xylose.

9. A method for producing xylitol, the method comprising:
culturing the transformant of claim 4 in a substrate containing xylose (stage 1); and
obtaining the xylitol from the culture cultured in stage 1 (stage 2).

10. The method of claim 9, wherein the transformant is E. coli W3310 ΔxylAB pTRCHIS2A-yahK of strain accession number KCTC15024BP.

11. The method of claim 9, wherein the substrate in stage 1 further contains glycerol.

12. The method of claim 11, wherein the glycerol is contained in an amount of 0.2 to 0.8% (v/v).

13. A composition for producing xylitol comprising the transformant of claim 4.
